(19) United States
(12) Patent Application Publication
(10) Pub. No.: US 2025/0107783 A1
(43) Pub. Date: Apr. 3, 2025

(54) SYSTEM, DEVICE, AND METHOD OF A ONE-HANDED ASPIRATOR

(71) Applicant: University of Virginia Patent Foundation, Charlottesville, VA (US)

(72) Inventors: Mark D. Miller, Crozet, VA (US); Ian A. Backlund, Charlottesville, VA (US)

(21) Appl. No.: 18/902,235

(22) Filed: Sep. 30, 2024

Related U.S. Application Data
(60) Provisional application No. 63/541,695, filed on Sep. 29, 2023.

(51) Int. Cl. A61B 10/00

(52) U.S. Cl. CPC A61B 10/0045; A61B 2217/005; A61B 2560/0425

(57) ABSTRACT

Provided are embodiments of a device, having a device body suitable to hold a syringe. The device body can have an elongated member with a distal support and a proximal support. The device further includes a ratcheting rod configured to fit moveably within an open chamber in the elongated member, where the ratcheting rod has a proximal end configured to connect to a plunger of the syringe. The device can include a lever handle hingedly connected to a distal end of the device body, and a ratcheting lever hingedly connected to a distal end of the lever handle and positioned to interact with the ratcheting rod. Compression of the lever handle forces the ratcheting lever to extend the ratcheting rod in a proximal direction and to extract the plunger of the syringe.

### CROSS-REFERENCE TO RELATED APPLICATION

This application claims priority to, and the benefit of, co-pending U.S. Provisional Patent Application entitled “System, Device, and Method of a One-Handed Knee Aspirator Device,” having Ser. No. 63/541,695, filed Sep. 29, 2023, which is hereby incorporated by reference in its entirety.

### BACKGROUND

Needle aspiration procedures are used as a diagnostic tool as well as a treatment tool for some conditions. For example, joint aspirations can be used to diagnose and treat various conditions in the joints. When a practitioner is performing a joint aspiration, it is often necessary to hold the joint in the proper position, stabilize the needle, and pull the plunger on the syringe to extract fluids or other materials. In some cases, it may also be necessary for a practitioner to manipulate the affected area in order to extract additional materials.

### SUMMARY

In accordance with the purpose(s) of this disclosure, as embodied and broadly described herein, the disclosure, in various aspects, relates to systems and devices for a one-handed aspirator and methods of use thereof. The solutions described herein combine various aspects of biomedical engineering, biology, medicine, and mechanical technologies to arrive at novel solutions for a one-handed aspirator device.

Aspects of the present disclosure provide for, among other things, a squeeze motion function, similar to a bike brake handle, or other squeeze motion. The handle squeeze component is coupled to a ratchet mechanism, which pushes a plunger of a syringe to an extended position, thereby creating suction. Embodiments of the present disclosure include: a device having a device body suitable to hold a syringe, where the device body has an elongated member with a distal support and a proximal support. In addition, the device can include a ratcheting rod configured to fit moveably within an open chamber in the elongated member, where the ratcheting rod has a proximal end configured to connect to a plunger of the syringe. The device can further include a lever handle hingedly connected to a distal end of the device body and a ratcheting lever hingedly connected to a distal end of the lever handle and positioned to interact with the ratcheting rod. In some examples, compression of the lever handle forces the ratcheting lever to extend the ratcheting rod in a proximal direction and to extract the plunger of the syringe.

In some examples, the syringe is a 60 CC syringe. In some examples, the ratcheting rod comprises a plurality of ratcheting teeth along a first side of the ratcheting rod. The first side of the ratcheting rod can be exposed through the open chamber in the elongated member. The lever handle can have grooves configured to fit fingers of a hand.

Additionally, the lever can be pumped repeatedly to modulate a volume pulled by the syringe. The syringe can be removed by disconnecting the proximal end of the ratcheting rod from the plunger of the syringe and removing the syringe from the device body. In some examples, unlocking the ratcheting lever releases the ratcheting rod back to a starting position.

Additionally, a method is provided for aspiration, including the steps of placing a syringe in a device body suitable to hold a syringe, where the device body has an elongated member with a distal support and a proximal support, and inserting a ratcheting rod in a chamber of the elongated member of the device body. The method can include twisting a plunger attachment on the ratcheting rod into a locked position causing the plunger attachment to attach to a plunger of the syringe and applying pressure on a lever handle hingedly connected to the elongated member of the device body, where the lever handle is coupled to the ratcheting rod. Compression of the lever handle transmits force through the ratcheting rod to raise the plunger of the syringe.

In some examples, the syringe comprises a 60 CC syringe. In some examples, wherein applying pressure on the lever handle comprises repeatedly pumping the lever handle to modulate a volume pulled by the syringe. The method can further include twisting the plunger attachment of the ratcheting rod into an unlocked position causing the plunger attachment to detach from the plunger of the syringe and allow removal of the syringe from the device body. In some examples, twisting the plunger attachment into an unlocked position releases the ratcheting rod back to a starting position. In some examples, placing the syringe in the device body further comprises twisting the syringe such that a flange of the syringe locks into a receiving slot on the device body.

Also provided is a system for aspiration having a syringe and a one-handed aspirator device. The device can include a device body suitable to hold the syringe and a ratcheting mechanism including a ratcheting rod disposed within the device body and a ratcheting lever. The device can include a lever handle on an exterior of the device body coupled to the ratcheting lever. The ratcheting rod, when in a locked position, can be attached to a plunger of the syringe. Compression of the lever handle forces the ratcheting lever to extend the ratcheting rod and to extract the plunger of the syringe.

In some examples, the syringe is a 60 CC syringe. In some examples, the ratcheting mechanism is linear. The lever handle can have grooves configured to fit fingers of a hand. The lever handle can be pumped repeatedly to modulate a volume pulled by the syringe. The syringe can be removed by unlocking the ratcheting rod and pulling the syringe out of the device body. In some examples, unlocking the ratcheting rod releases the ratchet mechanism back to a starting position.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an illustration of an exploded view of a system for one-handed aspiration according to various embodiments of the present disclosure.

FIGS. 2A and 2B are illustrations of an assembled device for one-handed aspiration according to various embodiments of the present disclosure.

FIGS. 3A-3C show different views of an assembled device for one-handed aspiration according to various embodiments of the present disclosure.

FIGS. 4A-4C show different views of a ratcheting rod of the device of FIG. 1 according to various embodiments of the present disclosure.

FIGS. 5A-5C show different views of a ratcheting lever of the device of FIG. 1 according to various embodiments of the present disclosure.

FIGS. 6A-6C show different views of a body of the device of FIG. 1 according to various embodiments of the present disclosure.

FIGS. 7A-7C show different views of a plunger attachment of the device of FIG. 1 according to various embodiments of the present disclosure.

FIGS. 8A-8C how different views of a lever of the device of FIG. 1 according to various embodiments of the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, biomedical engineering, mechanical engineering, medical techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Discussion

Disclosed are various approaches for a one-handed syringe aspirator device and methods of use thereof. Currently, when the medical procedure of aspiration is performed with a syringe, is a cumbersome process which often requires more than one person to stabilize the needle, hold the body of the syringe, and withdraw the syringe plunger to perform the aspiration. In the case of knee or other joint aspirations, yet another hand is necessary in order to “milk” the joint, or manually manipulate fluid surrounding the joint. It can be difficult for a single practitioner to manage these tasks all at once, thus additional practitioners may be necessary. This increases difficulty of the procedure as well as cost, time, and potentially discomfort for the patient.

Accordingly, various embodiments of the present disclosure are directed to systems and methods for a one-handed aspirator device. The device can have a body which is suitable to receive and stabilize a syringe. In addition, the device can have a ratcheting rod which fits into the device body and is secured to the syringe by means of a plunger attachment. The device has a lever which can include a ratcheting lever. Thus, when the lever is squeezed toward the body of the device, the ratcheting lever forces the ratcheting rod and the attached plunger in an outward direction from the body of the syringe. This creates aspiration.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

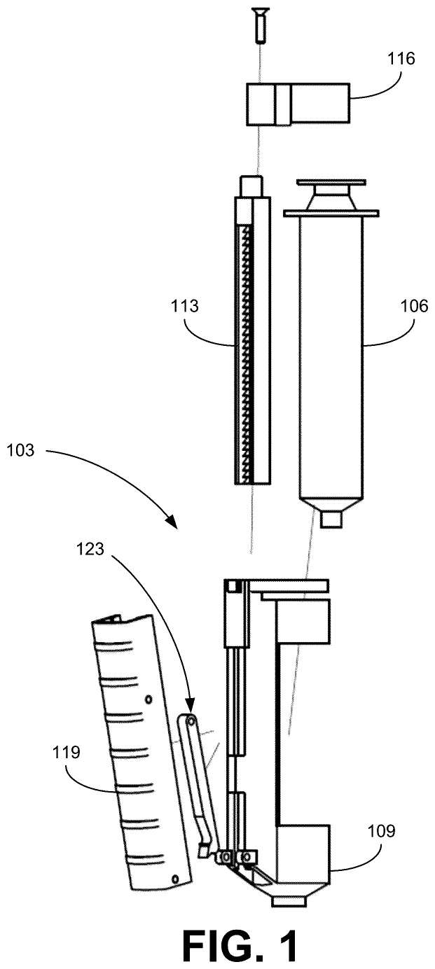

With reference to FIG. 1, shown is an exploded view of a one-handed aspirator system 100 having an aspirator device 103 and a syringe 106. The syringe 106 can be inserted into the body 109 of the aspirator device 103. Once inserted, the syringe 106 can be locked into place. The aspirator device 103 also includes a ratcheting rod 113 which fits into the body 109 of the aspirator device 103. At a top end of the ratcheting rod 113, a plunger attachment 116 can be included to lock onto the plunger of the syringe. The plunger attachment 116 can be rotatable about an axis parallel to the length of the syringe 106. In some examples, the plunger attachment 116 includes a receiving chamber for the flange at the end of the syringe 106 such that when the plunger attachment 116 is rotated toward the plunger of the syringe 106, the flange of the plunger fits within the receiving chamber of the plunger attachment 116. The aspirator device 103 can further include a lever 119. The lever 119 can be hingedly attached to the body 109 of the aspirator device 103. When the lever 119 is squeezed toward the body 109, the lever 119 can force a ratcheting lever 123 to drive the ratcheting rod 113 in a direction away from the body 109.

The aspirator device 103 can be comprised of a plastic, rigid polymer, metal, metal alloy, or other rigid material or combination of materials. In some examples, the aspirator device 103 can be disposable and is made of one or more disposable materials. However, in some examples, the aspirator device 103 is reusable and made from one or more materials which can be easily sterilized.

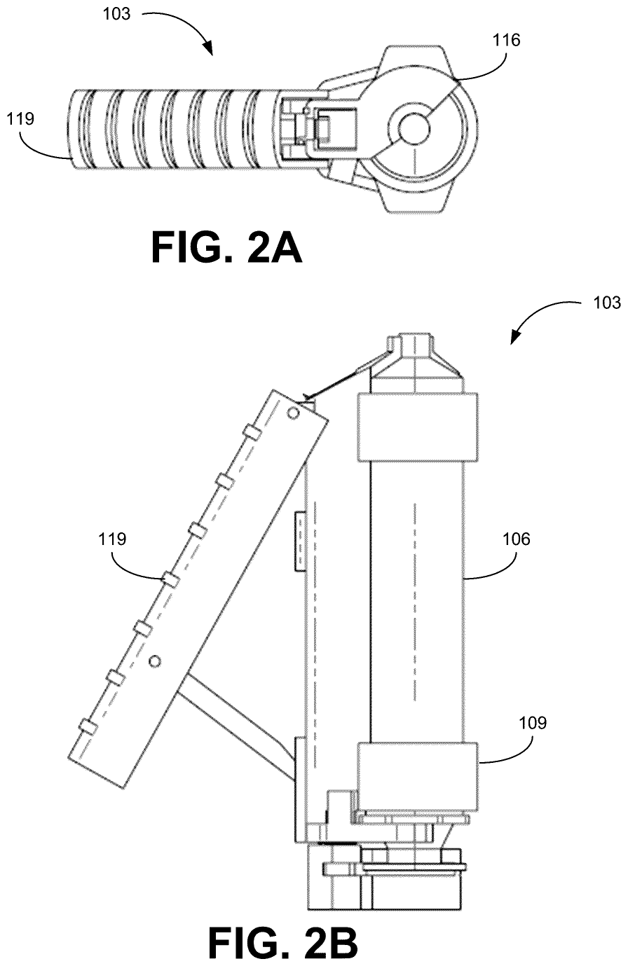

Moving to FIG. 2A, shown is a top view of an assembled one-handed aspirator device 103. In this view, the lever 119 is extended and the plunger-end of the syringe 106 is visible. As can be appreciated in FIG. 2A, the plunger attachment 116 can be fitted around a portion of the flange of the plunger, in order to exert force upon the plunger when the lever 119 is compressed. Also shown in FIG. 2A, the lever 119 can include one or more ridges to improve grip. In some examples, the lever 119 has a plurality of ridges configured to fit the fingers of a hand. Each ridge can be spaced apart about the average width of a finger.

In FIG. 2B, shown is a side view of an assembled one-handed aspirator device 103 with a syringe 106. From this view, the connection of the lever 119 to the device body 109 is shown. As previously described, the lever 119 can be hingedly connected to the device body 109 at the distal end or the end where a needle connects to the syringe 106. This placement of the hinged connection can improve stability of the needle end of the aspirator device 103 when the lever 119 is squeezed and operated.

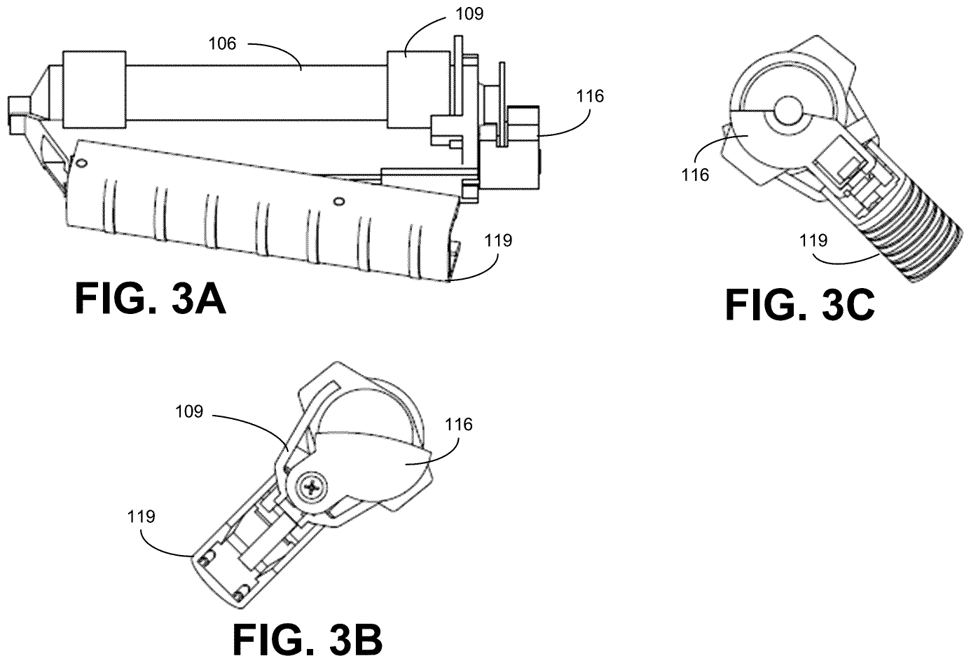

Next, FIGS. 3A, 3B, and 3C shown a variety of views of the assembled aspirator device 103. FIG. 3A shows a side view of the aspirator device 103. This view shows the plunger attachment 116 fitting around the flange of the plunger. When the syringe 106 is loaded into the device body 109, the plunger attachment 116 is in a first position which leaves space for the syringe 106 to be loaded. After loading the syringe 106, the plunger attachment 116 can be rotated to a second position (shown in FIG. 3A). In the second position, the plunger attachment 116 is secured to the plunger of the syringe 106. FIG. 3B shows a top view of the assembled aspirator device 103. As shown in FIG. 3B, the plunger attachment 116 can be connected to the device body 109 by means of a screw, bolt, rivet, or other fastener.

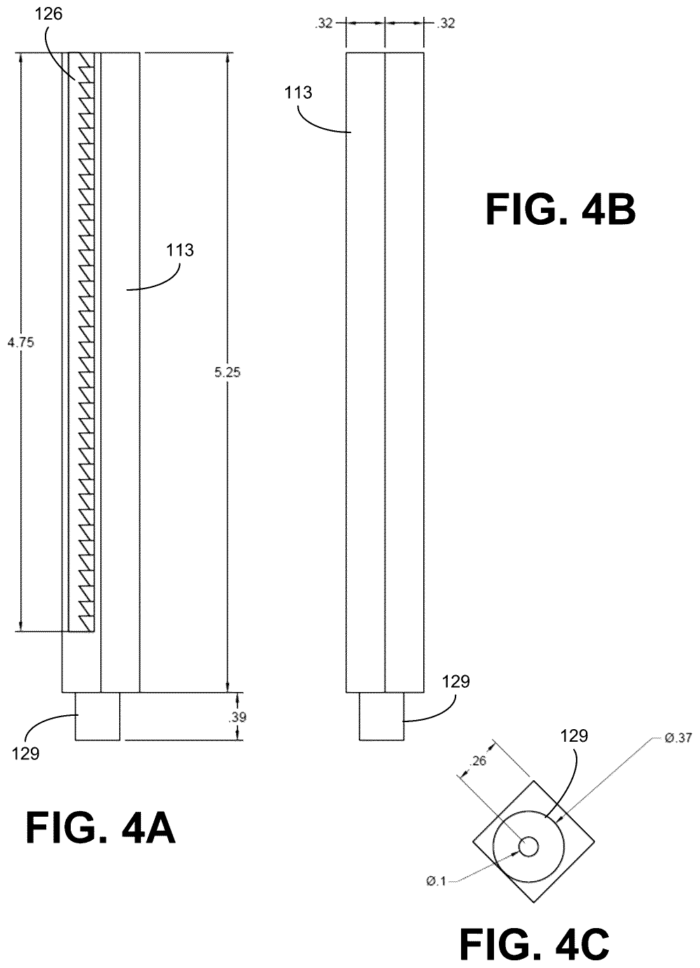

In FIG. 4A, shown is an example side view of a ratcheting rod 113. In some embodiments, the ratcheting rod 113 can have a plurality of sides. For example, as shown in FIGS. 4A-4C, the ratcheting rod 113 can be rectangular in shape, having four sides. However, in other embodiments, the ratcheting rod 113 can have another shape such as cylindrical, triangular, pentagonal, hexagonal, etc. On one side of the ratcheting rod 113, there can be a plurality of ratcheting teeth 126. Each ratcheting tooth 126 can comprise a wedge-shaped structure allowing unidirectional motion of the ratcheting lever 123. In some examples, the ratcheting rod 113 can include a connection point 129 at a proximal end which is configured to attach to the plunger attachment 116. The connection point 129 can be configured to receive a screw, bolt, rivet, or other fastener. FIG. 4B shows another example side view of the ratcheting rod 113, where the ratcheting teeth 126 are not pictured. Similarly, FIG. 4C shows a top view of the ratcheting rod 113, with a focus on the connection point 129. As shown in each of the examples of FIGS. 4A-4C, the ratcheting rod 113 can have certain dimensions in order to fit within a device made for a 60 CC syringe. In at least one example, shown in FIGS. 4A, the ratcheting rod 113 can have a length of about 5.25 inches, with the connection point 129 adding about 0.39 inches, and the length of the strip of ratcheting teeth 126 being about 4.75 inches. In the example of FIG. 4B, the sides of the ratcheting rod 113 can each have a width of about 0.32 inches. In the example of FIG. 4C, the connection point 129 can have an exterior diameter of approximately 0.37 inches and an interior diameter of approximately 0.1 inch. In some examples, the center of the connection point 129 can be offset from an edge of the ratcheting rod 113 by about 0.26 inches. However, various other measurements and configurations are possible for different sizes of syringes, etc.

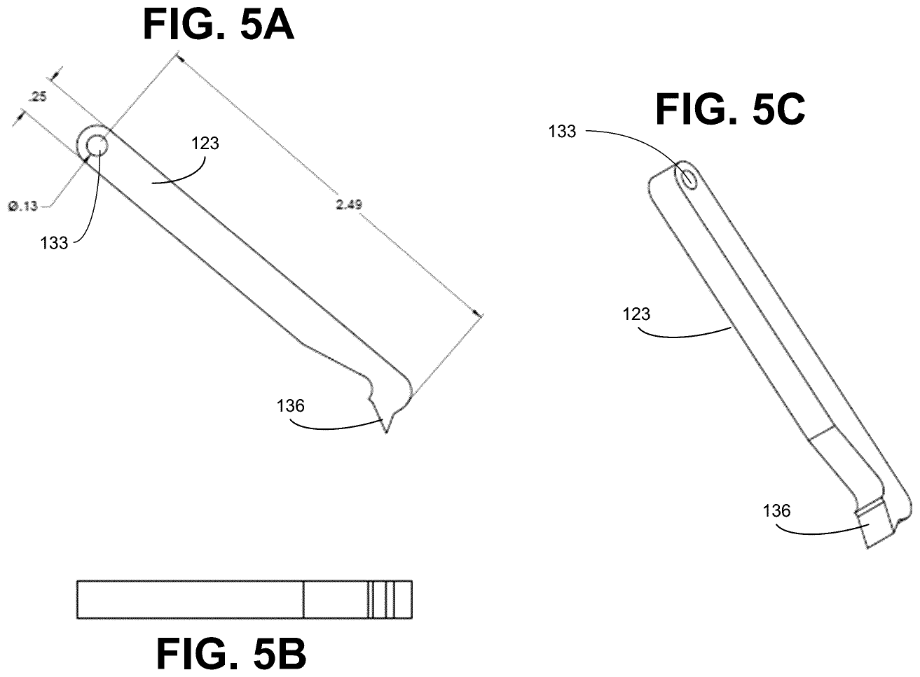

Next, in FIG. 5A, shown is an example side view of a ratcheting lever 123. FIG. 5B shows a bottom view of the ratcheting lever 123, and FIG. 5C shows a perspective view of the ratcheting lever 123. The ratcheting lever 123 can be configured to have a hinged connection 133 on a first end to connect to the lever 119. In some examples, the hinged connection 133 can comprise a hole and a pin. On the other end, the ratcheting lever 123 can have a foot 136. The foot 136 can comprise a sloped member which is suited to fit between the ratcheting teeth 126 on the ratcheting rod 113. In some embodiments, the foot 136 is a hook-shape, but in other examples, the foot 136 is flat, wedged, straight, pointed, or another shape which is suitable to fit between the ratcheting teeth 126. As shown in the example of FIG. 5A, the ratcheting lever 123 can be approximately 0.25 inches wide. The diameter of the hinged connection 133 can be approximately 0.13 inches. Similarly, the example of FIG. 5A shows that the center point of the hinged connection 133 can be approximately 2.49 inches from the foot 136.

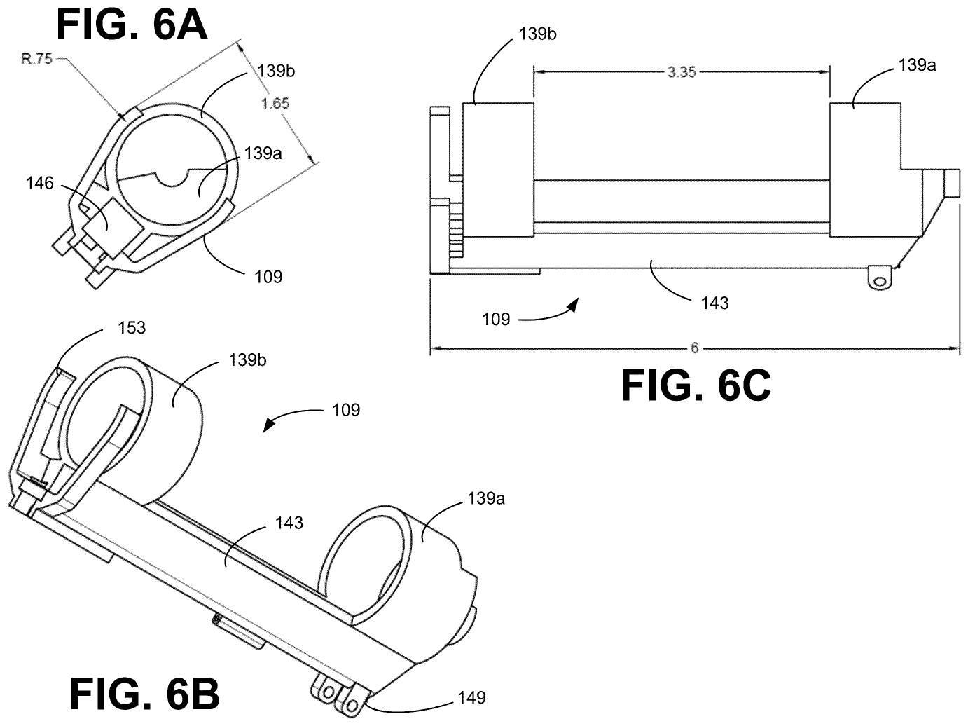

Moving to FIG. 6A, shown is a top view of the device body 109. The body 109 can include one or more syringe supports 139. In some examples, as shown in FIGS. 6B and 6C, the body 109 can include a first syringe support 139a and a second syringe support 139b. The first syringe support 139a can be located at a distal end of the body 109 and can be configured to receive the needle end of the syringe 106. In some examples, a needle can be attached to the syringe 106 through a suitable opening in the first syringe support 139a. The syringe 106 can be loaded into the device body 109 through the second syringe support 139b. The device body 109 can further include an elongated member 143 which stabilizes the syringe 106 and connects the first syringe support 139a to the second syringe support 139b. As shown in FIG. 6A, the elongated member 143 can have an open chamber 146 configured to receive the ratcheting rod 113. In addition, the elongated member 143 can include a lever attachment 149 for attaching to the lever 119. The body 109 can further include a syringe locking mechanism 153 near the second syringe support 139b at a proximal end. The syringe locking mechanism 153 can consist of a first arm and a second arm which extend from the elongated member 143 toward the syringe 106. The first arm and the second arm of the syringe locking mechanism 153 can be spaced from a top edge of the second syringe support 139b such that the flange of the syringe 106 can fit between the arms and the second syringe support 139b. Thus, when a syringe 106 is loaded into the device 103, the syringe 106 can be twisted such that the flange is fitted securely between the arms of the syringe locking mechanism 153 and the second syringe support 139b.

In some examples, the body 109 is approximately 6 inches long, with a distance between the first syringe support 139a and the second syringe support 139b being approximately 3.35 inches long. The body 109 can have a total width of approximately 1.65 inches.

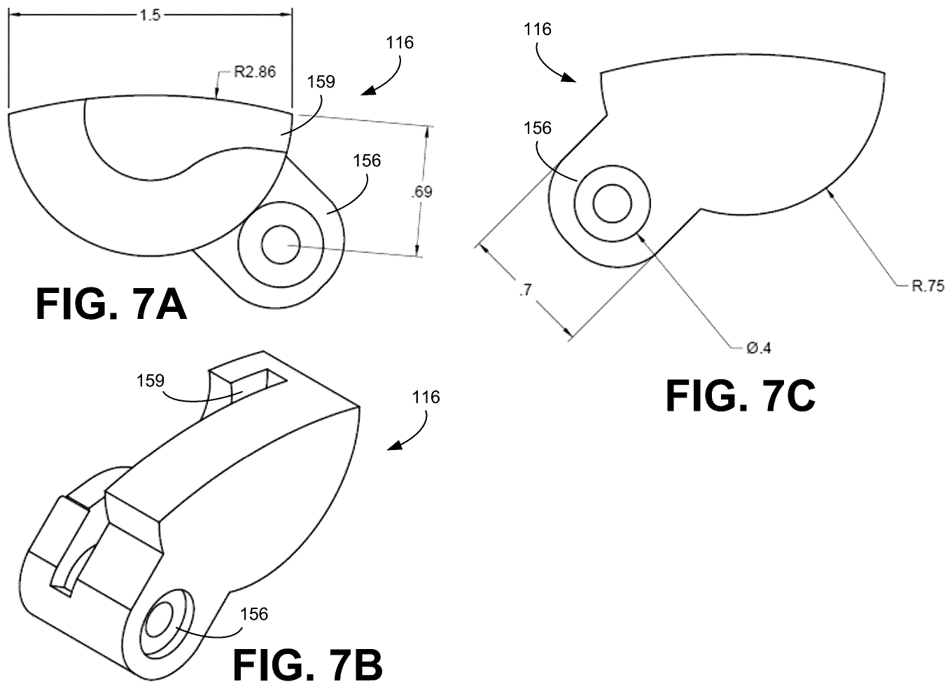

Next, at FIG. 7A, shown is a bottom view of the plunger attachment 116. The plunger attachment 116 can include an attachment region 156 for connecting the plunger attachment 116 to the proximal end of the device body 109. The attachment region 156 can be configured to receive a screw, bolt, rivet, rod, or other fastener to connect the plunger attachment 116 to the proximal end of the device body 109. In some examples, the plunger attachment 116 is rotatable around the connection means at the attachment region 156. As shown in FIG. 7B, a perspective view of the plunger attachment 116, the plunger attachment 116 can further include a receiving chamber 159. The receiving chamber 159 can be configured to fit around at least a portion of the flange of the syringe plunger. FIG. 7C shows a top view of the plunger attachment 116. According to various examples, the diameter of a hole in the attachment region 156 is approximately 0.4 inches, while the width of the attachment region 156 can be approximately 0.7 inches. The plunger attachment 116 can have a radial curve similar to that of a circle with a radius of 0.75 inches, which can vary corresponding to the size of the plunger of the syringe 106.

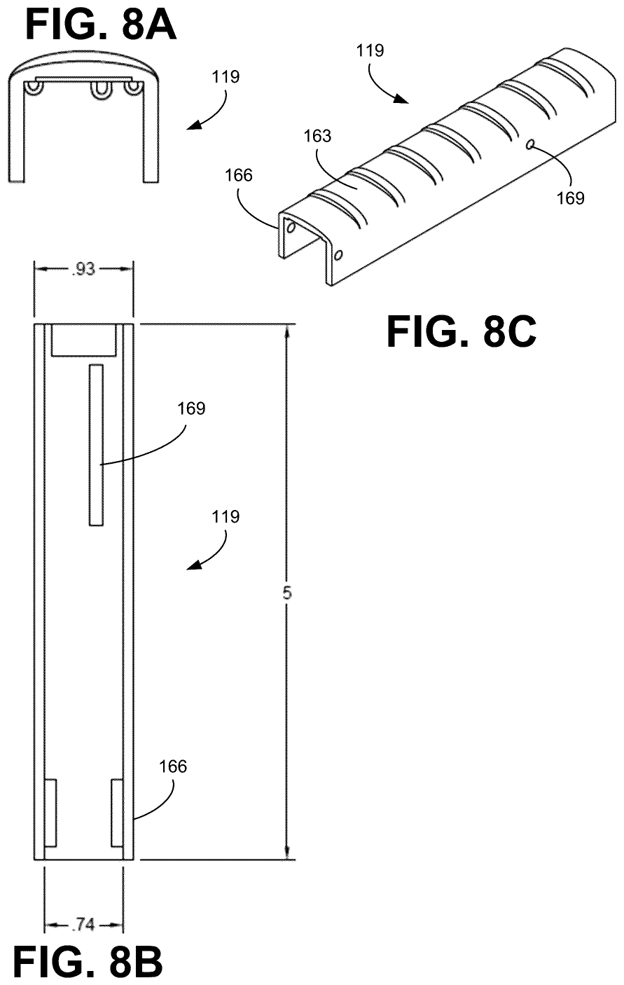

Moving to FIG. 8A, shown is a top view of the lever 119. FIG. 8B shows a side view of the lever 119, and FIG. 8C shows a perspective view of the lever 119. The lever 119 can include a handle or grip portion 163 on an exterior side of the lever 119. The grip portion 163 can include one or more ridges to improve grip, or in some examples, can include a sticky, cushioned, or rough material to improve grip. The lever 119 can include a body attachment 166 for connecting to the lever attachment 149 of the body 109. In some examples, the lever 119 can include a ratcheting lever attachment 169 positioned a distance from the distal end of the lever 119. In some examples, the lever 119 can be approximately 5 inches in length, with an exterior width of approximately 0.93 inches and an interior width of approximately 0.74 inches.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A device, comprising:
a device body suitable to hold a syringe, the device body having an elongated member with a distal support and a proximal support;
a ratcheting rod configured to fit moveably within an open chamber in the elongated member, the ratcheting rod having a proximal end configured to connect to a plunger of the syringe;
a lever handle hingedly connected to a distal end of the device body;
a ratcheting lever hingedly connected to a distal end of the lever handle and positioned to interact with the ratcheting rod; and
wherein compression of the lever handle forces the ratcheting lever to extend the ratcheting rod in a proximal direction and to extract the plunger of the syringe.

2. The device of claim 1, wherein the syringe is a 60 CC syringe.

3. The device of claim 1, wherein the ratcheting rod comprises a plurality of ratcheting teeth along a first side of the ratcheting rod, and wherein the first side of the ratcheting rod is exposed through the open chamber in the elongated member.

4. The device of claim 1, wherein the lever handle has grooves configured to fit fingers of a hand.

5. The device of claim 1, wherein the lever can be pumped repeatedly to modulate a volume pulled by the syringe.

6. The device of claim 1, wherein the syringe can be removed by disconnecting the proximal end of the ratcheting rod from the plunger of the syringe and removing the syringe from the device body.

7. The device of claim 1, wherein unlocking the ratcheting lever releases the ratcheting rod back to a starting position.

8. A method for aspiration, comprising:
placing a syringe in a device body suitable to hold a syringe, the device body having an elongated member with a distal support and a proximal support;
inserting a ratcheting rod in a chamber of the elongated member of the device body;
twisting a plunger attachment on the ratcheting rod into a locked position causing the plunger attachment to attach to a plunger of the syringe; and
applying pressure on a lever handle hingedly connected to the elongated member of the device body, the lever handle being coupled to the ratcheting rod,
wherein compression of the lever handle transmits force through the ratcheting rod to raise the plunger of the syringe.

9. The method of claim 8, wherein the syringe comprises a 60 CC syringe.

10. The method of claim 8, wherein applying pressure on the lever handle comprises repeatedly pumping the lever handle to modulate a volume pulled by the syringe.

11. The method of claim 8, further comprising:
twisting the plunger attachment of the ratcheting rod into an unlocked position causing the plunger attachment to detach from the plunger of the syringe; and
removing the syringe from the device body.

12. The method of claim 11, wherein twisting the plunger attachment into an unlocked position releases the ratcheting rod back to a starting position.

13. The method of claim 11, wherein placing the syringe in the device body further comprises twisting the syringe such that a flange of the syringe locks into a receiving slot on the device body.

14. A system for aspiration, comprising:
a syringe; and
a one-handed aspirator device comprising:
a device body suitable to hold the syringe;
a ratcheting mechanism including a ratcheting rod disposed within the device body and a ratcheting lever; and
a lever handle on an exterior of the device body coupled to the ratcheting lever,
wherein the ratcheting rod, when in a locked position, is attached to a plunger of the syringe, and
wherein compression of the lever handle forces the ratcheting lever to extend the ratcheting rod and to extract the plunger of the syringe.

15. The system of claim 14, wherein the syringe is a 60 CC syringe.

16. The system of claim 14, wherein the ratcheting mechanism is linear.

17. The system of claim 14, wherein the lever handle has grooves configured to fit fingers of a hand.

18. The system of claim 14, wherein the lever handle can be pumped repeatedly to modulate a volume pulled by the syringe.

19. The system of claim 14, wherein the syringe can be removed by unlocking the ratcheting rod and pulling the syringe out of the device body.

20. The system of claim 14, wherein unlocking the ratcheting rod releases the ratchet mechanism back to a starting position.
